(19) United States
(12) Patent Application Publication
(10) Pub. No.: US 2009/0177263 A1
(43) Pub. Date: Jul. 9, 2009

(54) ENFORCED GUIDING CATHETER

(76) Inventor: Alexander G. VILLER, Moscow (RU)

Correspondence Address: BARDMESSER LAW GROUP, P.C., 1025 CONNECTICUT AVENUE, N.W., SUITE 1000, WASHINGTON, DC, 20006, US

(21) Appl. No.: 12/397,364

(22) Filed: Mar. 4, 2009

Related U.S. Application Data
(63) Continuation-in-part of application No. PCT/RU2006/000469, filed on Sep. 4, 2006.

(51) Int. Cl. A61F 2/84; A61M 25/10

(52) U.S. Cl. 623/1.11; 604/98.01

(57) ABSTRACT

A guiding catheter for endovascular recanalization of occluded coronary arteries includes a polymeric tube having a main channel, for introduction of a dilatation balloon catheter or a stent along the guide. The polymeric tube has an auxiliary channel for providing overpressure to an elastic balloon(s) that is located on a distal end of the catheter. The elastic balloon(s) is in communication with the auxiliary channel and has at least one fixating protrusion on its outer surface. The catheter can have between 2 and 4 elastic balloons at its distal end, each elastic balloon having at least one fixating protrusion on its corresponding outer surface. The fixating protrusion is preferably in a form of a hemisphere, or is elongated in a direction of the longitudinal axis of the catheter. A height of the fixating protrusion is between 0.01 and 1 mm above the surface of the elastic balloon. A proximal end of the catheter includes a Y-shaped adapter for introduction of radiopaque agent or instruments into a first input of the Y-shaped adapter, and a second input of the Y-shaped adapter is connectable to a high pressure syringe capable of inflating the balloon(s).

### CROSS-REFERENCE TO RELATED APPLICATIONS

This application is a continuation-in-part of PCT/RU2006/000469, filed on 4 Sep. 2006, which is incorporated herein by reference.

### BACKGROUND OF THE INVENTION

1. Field of the Invention

The present invention is related to medical technologies and can be applied in endovascular surgery and in interventional cardiology, such as for recanalization of chronically occluded coronary arteries.

2. Description of the Related Art

Delivery of instruments or a pharmaceutical composition immediately to a place of a vascular disruption by application of guiding catheters is commonly known in the field of endovascular surgery, including interventional cardiology. The guiding catheter, as a rule, is in the form of an elastic tube made of a smooth polymer and a radiopaque tip, usually, reinforced to ensure the necessary flexibility for minimization of trauma to the vascular walls. After positioning a guiding catheter at a location of prospective operation the working instrument or a medical product is delivered through the inner channel of the catheter to the place of operation (see, e.g., patent RU2203104). The position of the distal end of a guiding catheter is usually controlled by means of observation of its contrast level or of its radiopaque labels.

However, the outer diameter of a guiding catheter is, as a rule, substantially smaller than the inner diameter of the blood-vessel into which the catheter is inserted. This enables elimination of possibility of occlusion of the blood-vessel and of interruption of the blood flow. However, a drawback of such design is insufficient fixation of a guiding catheter in a lumen of the arterial segment, seriously complicating or restricting efficiency of the operation, which is performed by instruments introduced through the catheter.

Used in intervention cardiology in the course of balloon angioplasty and coronary stenting, the guiding catheters are fixed in lumens of coronary arteries by using special flexures, and also due to their introduction beyond the arterial ostium, which can result in a high risk of a mechanical trauma of an arterial wall, its dissection and/or thrombosis. Rigid bracing of a guiding catheter in lumen of an artery constitutes a necessary condition for conducting a recanalization of a chronic arterial occlusion, since the inserted instruments need to overcome resistance in the occluded segment.

There are several methods of improving bracing of a guiding catheter: 1) insertion of an additional coronary wire; 2) insertion of a balloon catheter in an artery at the moment of the recanalization process by a special wire; 3) insertion of an additional wire in a lateral branch of an artery in a location more proximal than an occlusion area, insertion of a balloon catheter along this wire and inflation into the side branch, where the balloon catheter serves as an “anchor”.

At the same time, the above methods do not solve, to the full extent, the problem of bracing of a guiding catheter in an arterial lumen, and anatomical diversification of arteries' shapes do not always permit implementing these methods. They also necessitate the use of additional tools and are, in many cases, technically impossible. Furthermore, despite introduction, in the clinical practice, of new coronary wires with increased rigidity, and also various apparatus methods of a recanalization of chronic arterial occlusions (excimer laser, SafeCross system, etc.), fixation of a guiding catheter in an arterial lumen remains the major and still unsolved aspect of endovascular recanalization of chronic arterial occlusions.

Devices for bracing (or “anchoring”) the distal end of a guiding catheter are known, for example, see U.S. Pat. No. 5,885,238 in which the system of endovascular blocking of a coronary artery lumen by means of an inflatable balloon at the distal end of the catheter, is described. In the described system, the catheter is not a guiding catheter, but only provides “a gag”, blanking coronary arteries, i.e., a different problem is solved; however, the principle of an inflatable balloon is applicable to bracing (fixation) the distal end of a guiding balloon.

This is described in, for example, the U.S. Pat. No. 5,000,743. This patent describes a system consisting of two catheters, i.e., a guiding catheter and a balloon dilatation catheter. The guiding catheter includes, at the distal end, an inflatable balloon located around the catheter, which, in an inflated state, provides a certain degree of fixation of the position of the distal end of the guiding catheter. Such fixation ensures improved accuracy of introduction of a working part of dilatation catheter in a blood vessel.

The main drawback of the above solution consists of practically a full closure of a blood vessel by the inflated balloon, though the authors have tried to solve this problem, at least in part, by disposing holes in the wall of the guiding catheter not far from the balloon. Such a solution, however, still severely impedes the blood flow in the blood vessel and, thus, forces a restriction as to the operation duration. Besides, as the guiding catheter is usually made of a material providing a sufficient slip in the blood vessel, the balloon with the same surface will be reliable fixed only at rather high inner pressure, which can result in a trauma of the blood vessel. When the tools introduced through such a catheter face resistance, the catheter can slip out of the lumen of an artery.

### SUMMARY OF THE INVENTION

The problem to be solved by the claimed invention consists in providing a reliable fixation of the distal end of a guiding catheter in a blood vessel with the minimal trauma of vascular walls, having maintained the sufficient channels for blood flow.

The technical result ensures stability of bracing of a catheter while preserving free blood circulation, which results in fewer homodynamic complications during operation, and also in better chance of success in endovascular recanalization of chronically occluded arteries.

The invention includes a guiding catheter for endovascular recanalization of chronically occluded coronary arteries, comprising a polymeric tube provided with a main channel capable of introducing a dilatation balloon catheter or a stent, and an auxiliary channel capable of building an overpressure in an elastic balloon located on the distal end of the catheter; the elastic balloon consists of at least one balloon located on the surface of the polymeric tube, which balloon is in communication with the auxiliary channel and is supplied, on its outer surface with at least one element of additional fixation. The balloon itself is preferably made of nylon, PTFE or PET, and/or other materials known in the art, and is typically between 2 and 30 mm long, and the cross-section size (height above the surface of the catheter) is in the range of 0.5 to 5 mm. If there is more than one protrusion, they are mainly located radially and symmetrically (equiangularly) relative to the longitudinal axis of the catheter.

The perimeter of a working surface of a balloon on the surface of the polymeric tube also preferably does not exceed half of the perimeter of the cross section of the polymeric tube.

To achieve the best traction of the inflated balloon with blood vessel walls, the element of the additional fixation is executed in the form of at least one fixing protrusion which can be preferably formed of the same material, as an elastic balloon. Preferably the fixating protrusion is in the form of a hemisphere. Other protrusion shapes are possible, for example, elongated or elliptical shapes, such as generally elongated in the same direction as the axis of the catheter, etc.

If the number of protrusions is more than one, the protrusions are preferably located symmetrically about the longitudinal axis of the polymeric tube (i.e., arranged equiangularly). Note that as an option, the protrusions may be variously distributed along the length of the balloon (i.e., they do not all need to be located at the same position on the length of the balloon, even if they are distributed equiangularly.) As a further option, there may be multiple rows of protrusions along the length of the balloons (i.e., 3 protrusions arranged equiangularly in the distal portion of the balloon, and 3 protrusions in the proximal portion). The angular arrangement of the sets of protrusions of each row may be aligned, or they may be offset from each other (i.e., in the case of 3-3 arrangement, row 1 is offset by 60 degrees from row 2, in the case of 2-2 arrangement, offset by 90 degrees, and so on). The protrusions may be formed of nylon, PTFE, PET, etc. (and are generally solid inside). The material of the protrusions may be the same as the material of the balloon, or may be made of the different material.

A typical height of the protrusion above the surface of the balloon is on the order of 0.01-1 mm (above the surface of the balloon), more preferably between 0.1 and 0.3 mm, more preferably still between 0.015 and 0.25 mm. Generally, if the protrusions are too large, they may damage the artery, and if they are too small, they have too insignificant of an effect. If the balloon has a lubricating coating for better sliding through the arteries, the coating may be applied to the protrusions as well, or the coating may be left off the protrusions. Typically, each balloon has at least one protrusion, and more preferably several (e.g., 2-5) protrusions. An optimal configuration is believed to be three balloons, arranged equiangularly (at 120 degrees relative to each other) about the longitudinal axis of the catheter. The balloons may be individually inflatable, or may be inflatable simultaneously

Overpressure in an elastic balloon is provided by controlled introduction of a radiopaque substance that increases accuracy of positioning of the distal end of a guiding catheter. Also, an overpressure in the elastic balloon is provided through taps to the auxiliary channel.

Preferably at the distal end of a catheter, the holes have been made for communication between the main channel and a blood flow in an aorta.

The proximal of a catheter can have the Y-adapter for injection, through one input of the basic channel, of a radiopaque substance, or for insertion of instruments, and the second input is connected to a high pressure syringe with capability of inflating the elastic balloon(s).

Additional features and advantages of the invention will be set forth in the description that follows, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE ATTACHED FIGURES

The accompanying drawings, which are included to provide a further understanding of the invention and are incorporated in and constitute a part of this specification, illustrate embodiments of the invention and together with the description serve to explain the principles of the invention.

FIG. 1 shows a general diagram of a claimed catheter.

FIGS. 2.1, 2.2 show a catheter located in an ostium of the coronal artery branching from aorta, and the mechanism of its fixation due to inflation of the balloon with a fixating protrusion (projection).

FIGS. 3.1, 3.2 show the cross section of artery in a place of location of a guiding catheter before inflation of balloon.

FIG. 4 shows the longitudinal section of guiding catheter.

FIG. 5 (5.1 to 5.6) demonstrate the operation of the claimed method in the process of endovascular recanalization of an occlusion of a coronary artery:

### DETAILED DESCRIPTION OF THE PREFERRED EMBODIMENTS

Reference will now be made in detail to the embodiments of the present invention, examples of which are illustrated in the accompanying drawings.

FIG. 1 shows a general diagram of a claimed catheter where 3 is the catheter, 11 is the Y-shaped proximal end, 5 is the distal end with the balloon.

FIGS. 2.1, 2.2 show catheter 3 located in an ostium of the coronal artery 2 branching from aorta 1, and the mechanism of its fixation due to inflation of balloon 5 with a fixating protrusion (projection) 9.

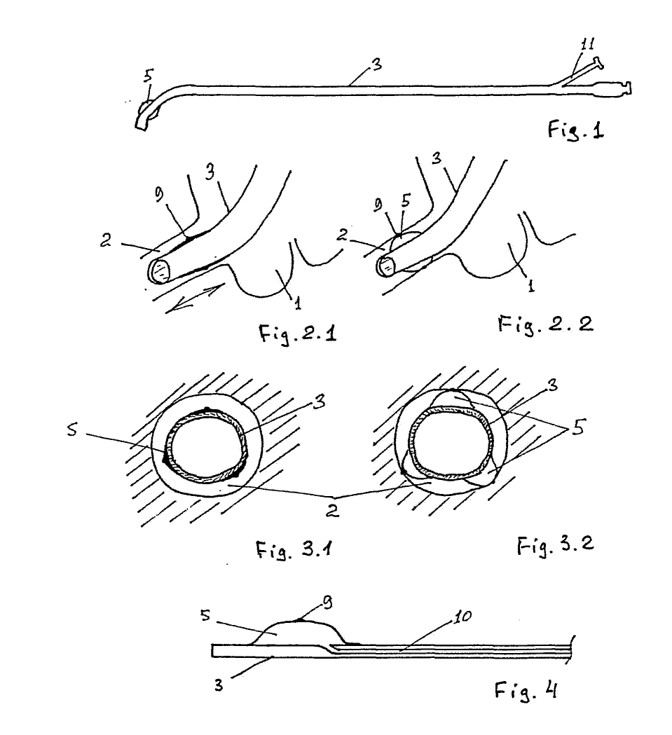

FIGS. 3.1, 3.2 show the cross section of artery 2 in a place of location of a guiding catheter before inflation of balloon 5 with balloons having the fixating protrusions, and after inflation of balloon 5.

The cross section shown on FIG. 3.2 allows seeing, in this example, the presence of three balloons 5, which provides many more opportunities for maintenance of practically normal blood flow. The holes described in the prototype, are kept intact in the proposed design, however, they do not play an essential role in maintenance of the blood flow at presence of wider channels shaped between the balloons and the arterial walls.

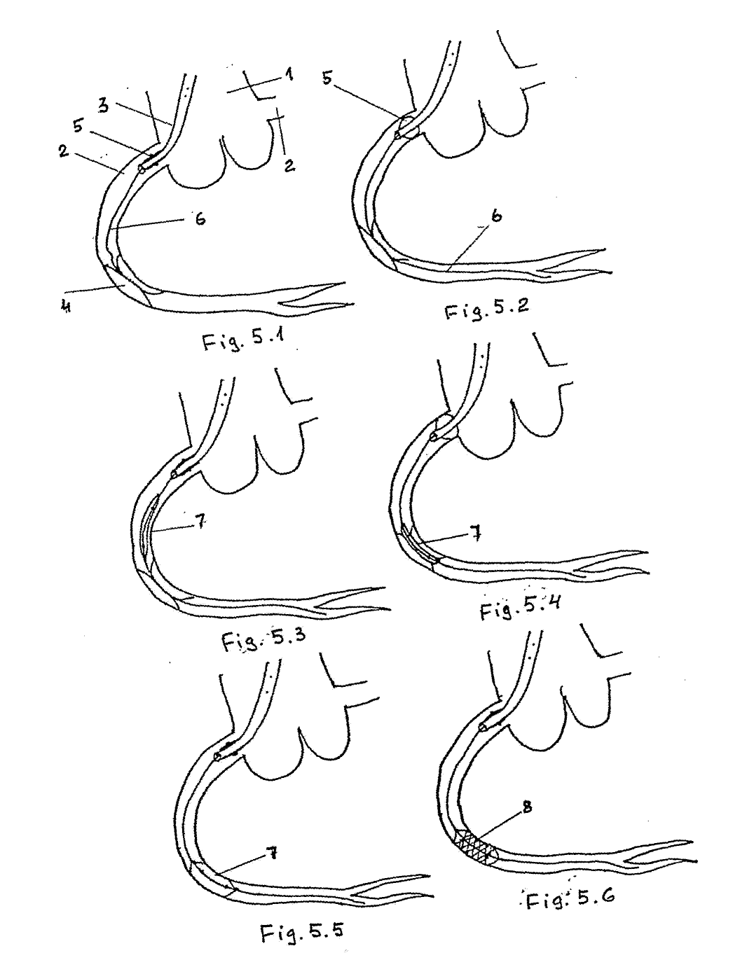

FIG. 4 shows the longitudinal section of guiding catheter 3, where the channel inside the walls of the catheter is schematically presented, leading to the balloon 5. Through this channel, and connected with a Y-shaped proximal end of the catheter, the balloon 5 is supplied by a contrast agent inflating the balloon, resulting in an expansion of the balloon and the guiding catheter is fixed in an artery by means of protrusions 9. Diagrams in FIG. 5 (5.1 to 5.6) demonstrate the operation of the claimed method in the process of endovascular recanalization of an occlusion of a coronary artery:
guiding catheter 3 is introduced into an ostium of the blood vessel 2;
in case of mechanical difficulties in guiding a coronary conductor 6 through an atherosclerotic occlusion 4, the balloons, at the distal end of a guiding catheter, are inflated by pumping the contrast agent, under pressure, in the balloon through the auxiliary channel, and, due to close contact of the balloon walls and fixating protrusions of the balloon and the arterial walls, the catheter is fixed in blood vessel 5 (FIG. 5.2);
further, the balloons can be deflated (FIG. 5.3) and inflated in case of difficulty in guiding balloon catheter 7 through occlusion area 4 (FIG. 5.4);
further, a routine balloon angioplasty of artery 2 in the occlusion area 4 is performed by means of balloon catheter 7 (FIG. 5.5) and, afterwards, an implantation of coronary stent 8 can be performed (FIG. 5.6).

Having thus described a preferred embodiment, it should be apparent to those skilled in the art that certain advantages of the described method and apparatus have been achieved. It should also be appreciated that various modifications, adaptations, and alternative embodiments thereof may be made within the scope and spirit of the present invention. The invention is further defined by the following claims.

What is claimed is:

1. A guiding catheter for endovascular recanalization of occluded coronary arteries comprising:
a polymeric tube having a main channel, for introduction of a dilatation balloon catheter or a stent along the guide, and
the polymeric tube further having an auxiliary channel for providing overpressure to an elastic balloon that is located on a distal end of the catheter,
wherein the elastic balloon includes at least one expansion volume located on outer surface of the polymeric tube, the expansion volume in communication with the auxiliary channel and having at least one fixating protrusion on its outer surface.

2. The guiding catheter of claim 1, wherein the catheter has between 1 and 4 elastic balloons at its distal end, each elastic balloon having at least one fixating protrusion on its corresponding outer surface.

3. The guiding catheter of claim 2, wherein the elastic balloons are located symmetrically relative to a longitudinal axis of the polymeric tube.

4. The guiding catheter of claim 3, wherein all the protrusions located on all the elastic balloons are located symmetrically relative to a longitudinal axis of the polymeric tube.

5. The guiding catheter of claim 3, wherein all the protrusions located on all the elastic balloons are located asymmetrically relative to a longitudinal axis of the polymeric tube.

6. The guiding catheter of claim 3, wherein the protrusions located on all the elastic balloons are arranged into multiple rows along the length of the catheter.

7. The guiding catheter of claim 1, wherein a length of the elastic balloon is between 1 and 30 mm and a cross-section of the elastic balloon is between 0.5 and 5 mm.

8. The guiding catheter of claim 7, wherein a length of a section of the elastic balloon perimeter on a surface of the polymeric tube does not exceed half of a perimeter of the cross-section of the polymeric tube.

9. The guiding catheter of claim 1, wherein the elastic balloon includes multiple protrusions on its surface.

10. The guiding catheter of claim 1, wherein the fixating protrusion is in a form of a hemisphere.

11. The guiding catheter of claim 1, wherein the fixating protrusion is elongated in a direction of the longitudinal axis of the catheter.

12. The guiding catheter of claim 1, wherein the fixating protrusion is formed of the same material as the elastic balloon.

13. The guiding catheter of claim 1, wherein the fixating protrusion is formed of a different material than the elastic balloon.

14. The guiding catheter of claim 1, wherein the elastic balloon acquires overpressure from controlled introduction of radiopaque material.

15. The guiding catheter of claim 1, wherein elastic balloon acquires overpressure through a pipe-bend of the auxiliary channel.

16. The guiding catheter of claim 1, wherein a height of the fixating protrusion is between 0.01 and 0.2 mm above the surface of the elastic balloon.

17. The guiding catheter of claim 1, wherein a height of the fixating protrusion is between 0.1 and 0.3 mm above the surface of the elastic balloon.

18. The guiding catheter of claim 1, wherein a proximal end of the catheter includes a Y-shaped adapter for introduction of radiopaque agent or instruments into a first input of the Y-shaped adapter, and wherein a second input of the Y-shaped adapter is connectable to a high pressure syringe capable of inflating the at least one expansion volume.
